(19) United States
(12) Patent Application Publication
(10) Pub. No.: US 2013/0266995 A1
(43) Pub. Date: Oct. 10, 2013

(54) METHOD FOR PRODUCING D-TYPE LACTIC ACID

(71) Applicants: Chae Hwan Hong, Ansan (KR); Si Hwan Kim, Anyang (KR); Jiyoun Seo, Suwon (KR); Do Suck Han, Seongnam (KR)

(72) Inventors: Chae Hwan Hong, Ansan (KR); Si Hwan Kim, Anyang (KR); Jiyoun Seo, Suwon (KR); Do Suck Han, Seongnam (KR)

(73) Assignee: HYUNDAI MOTOR COMPANY, Seoul (KR)

(21) Appl. No.: 13/597,728

(22) Filed: Aug. 29, 2012

(30) Foreign Application Priority Data
Apr. 4, 2012 (KR) 10-2012-0034861

(51) Int. Cl. C12P 7/56

(52) U.S. Cl. 435/139

(57) ABSTRACT

The present invention provides a method for producing D-type lactic acid, by a sugar fermentation of sugar. In accordance with the method of the invention, lactic acid produced during the fermentation reaction is selectively absorbed and detached using an active carbon stick. The method does not require steps, such as, lactate concentration and electrodialytic water splitting that are required during the lactic acid fermentation process. According to the invention, D-type lactic acid can be produced in a high purity and with a high yield. Further, the time and cost associated with the separation process can be reduced in a great extent, thereby offering advantageous effects from a commercial perspective.

### CROSS-REFERENCE TO RELATED APPLICATION

This application claims under 35 U.S.C. §119(a) the benefit of Korean Patent Application No. 10-2012-0034861 filed on Apr. 4, 2012, the entire contents of which are incorporated herein by reference.

### BACKGROUND

(a) Technical Field

The present provides a method for selectively producing D-type lactic acid by using active carbon stick.

(b) Background Art

Due to an increase in global population and rapid industrial development, the use of fossil fuel resources has rapidly increased. Consequently, problems, such as, global warming due to the green house gas emissions, and waste-attributed environmental pollution have been emerging as serious global issues.

A plant-based polymer (i.e., a biomass polymer) is a material produced by a chemical or biological method by using renewable plant resources, such as, corn, bean, sugar cane, wood, etc. The plant-based polymer is highly expected to serve its role in solving the environmental problems because of its reduced carbon dioxide emissions instead of biodegradability. Among the biomass polymers, poly-lactic acid or polylactide is a linear aliphatic polyester that can be obtained by fermentation of starch obtained from corn or potato. Alternately, poly-lactic acid can be obtained by neutralization of glycol monomers that are obtained through fermentation of plant-based cellulose after saccharification. Poly-lactic acid is a thermoplastic polymer with carbon-neutrality and eco-friendliness.

Currently, lactic acid that is used as a raw material for manufacturing poly-lactic acid is commercially produced by a petrochemical synthesis process and/or a bioengineering fermentation process. In the petrochemical synthesis process, lactic acid is produced through steps including oxidizing ethylene originated from crude oil to produce acetaldehyde; producing lacto-nitrile through an addition reaction of hydrogen cyanide; distilling and refining lacto-nitrile; and performing hydrolysis by using sulfuric acid or hydrochloric acid. However, lactic acid produced by the synthesis process is a racemic mixture, which contains 50 wt % of D-type lactic acid and 50 wt % of L-type lactic acid. In other words, a DL-type lactic acid is thus produced. Accordingly, the components of such lactic acid cannot be controlled.

On the contrary, in the bioengineering fermentation process, lactic acid is selectively produced by using a microorganism on a carbohydrate obtained in the nature, such as, starch or sugar cane. During the process, lactic acid is continuously generated through strain metabolism that occurs in a fermentation in a fermentation reactor. In other words, after the microorganism and a culture medium used for feeding the microorganism are added into the fermentation reactor, the strain discharges lactic acid when it is subject to the metabolism feeding in the culture medium. Lactic acid being produced here is a liquid acid, which in turn raises the acidity in the fermentation reactor. With the increased acidity in the fermentation reactor, the normal metabolism of the strain will be then disturbed. It is a disadvantage associated with the fermentation process, as the pH in the fermentation reactor needs to be adjusted to an appropriate level.

Regarding the lactic acid production process, Korean Pat. No. 10-0264714 discloses a method for processing a medium to recover lactic acid contained in the medium. The method includes steps of: absorbing lactic acid by contacting the medium containing lactic acid with a solid-phase and free-base divinylbenzene-crosslinked polymer that contains tertiary-amine function group; detaching lactic acid by using hot water at a temperature of about 75° C. or higher; and recovering a fraction containing essentially an aqueous solution of lactic acid. However, this method has certain disadvantages, such as, high manufacturing cost because the process of removing organic impurities is complicated, and the reaction is conducted at a high temperature.

Korean Patent Publication Application No. 10-2011-0076800 discloses a method of separating organic acid from fermentation broth, which includes steps of: (i) providing a supply material comprising a fermentation product which contains an organic acid; (ii) providing an ion exchange absorption medium; and (iii) eluting the supply material to a sorbent bed using the ion exchange absorption medium, thereby separating lactic acid from the supply material. In this method, the process is simple as lactic acid is separated through the ion exchange absorption medium. On the other hand, it is difficult to obtain lactic acid with a high purity by this method.

As mentioned herein above, lactic acid raises the acidity in the fermentation reactor during the fermentation process in producing lactic acid. Consequently, a basic neutralizing agent (such as, NaOH or NH₄OH) is added during the fermentation reaction to adjust the pH to 6.5. It follows that an acid-base reaction between the basic neutralizing agent and lactic acid that is being produced will occur to produce lactic acid as a final product of such a reaction in a lactate form (e.g., Na⁺Lac⁻ or LH₄⁺Lac⁻). One of the techniques for obtaining pure lactic acid from a salt-type lactate is described below.

The first example is a method of separating and concentrating lactate via electrodialysis. The second example is a method of converting lactate to lactic acid (Na⁺Lac⁻, NH₄⁺Lac⁻→Lac⁻H⁺) through electrodialytic water splitting. This method is complicated as it requires a two-step process, that is, the concentration of lactate and electrodialytic water splitting. Also, it requires a relatively long time, such as, about 72 hours, to perform the process.

Accordingly, there is an urgent need for the development of a technique for producing lactic acid with a reduced process time and a reduced manufacturing cost. Such a goal may be achieved by simplifying complicated process steps, such as, lactate concentration and electrodialytic water splitting.

The above disclosure in the Background section is intended only for a better understanding of the present invention. At no time, it may be construed as the Applicants' admission of the prior art that is well apprised by a person of ordinary skill in the art.

### SUMMARY OF THE DISCLOSURE

The present invention has been made in an effort to solve the above-described problems associated with the methods existing in the art.

The present invention provides a method for producing D-type lactic acid that can be used as a starting material to make poly-lactic acid. According to the present invention, lactic acid is produced in a high purity and with a high yield by a simple process, such as, a sugar fermentation.

In one aspect, the present invention provides a method for producing D-type lactic acid through fermentation of sugar, including steps of:
(a) adding a microorganism and a sugar component into a fermentation reactor to produce a fermentation liquor through fermentation;
(b) inserting a sterilized active carbon stick in the fermentation reactor to absorb lactic acid produced by the fermentation;
(c) separating from the fermentation reactor the active carbon stick with lactic acid absorbed therein and immersing the active carbon stick into a recovery reactor;
(d) detaching lactic acid from the active carbon stick in acetone contained in the recovery reactor, thereby to produce D-type lactic acid.

In an embodiment, the sugar is a mixture containing at least one selected from glucose, fructose, mannose, galactose and sucrose.

In another embodiment, the microorganism is selected from E. coli and Lactobacillus species, or a mixture thereof.

In still another embodiment, the fermentation in step (a) is performed at a temperature ranging between 37° C. and 45° C. and with a pH between 6.0 and 6.5.

In yet another embodiment, the fermentation in step (a) is performed by a batch process or a continuous process.

In still yet another embodiment, the step (B) is performed at a pH of 1˜5.5.

In a further embodiment, the active carbon stick is in a granule or a pellet type.

In another further embodiment, the active carbon stick has a pore size ranging between 0.1 μm and 0.5 μm.

In still another further embodiment, the volume of the active carbon stick in step (b) ranges from 10% to 20% of the volume of the fermentation liquor.

In yet another further embodiment, the step (c) is performed at a pH of 5.5˜6.5.

In still yet another further embodiment, the step (d) is performed at a pH of 1˜5.5.

In a still further embodiment, D-type lactic acid is recovered in a monomer state in step (d).

In a yet still further embodiment, the dwell time of the active carbon stick in steps (b) and (d) ranges between 30 minutes and 1 hour.

In a yet still further embodiment, the method further includes a repetitive operation, during which the active carbon stick with lactic acid detached in step (d) is sterilized and then added back to the fermentation reactor.

In the present invention, it is possible to produce D-type lactic acid with a high purity and a high yield by selectively absorbing and detaching lactic acid produced through a fermentation of a sugar component by using an active carbon stick.

Moreover, pure D-type lactic acid can be recovered during the lactic acid fermentation production process, without going through the lactate concentration and electrodialytic water splitting steps, thereby reducing the time and the cost associated with the separation process.

Other aspects and preferred embodiments of the invention are discussed infra.

### BRIEF DESCRIPTION OF THE DRAWINGS

The above and other features of the present invention are described herein in detail with reference to certain embodiments thereof as well as the illustration provided in the accompanying drawings. The embodiments and drawings are given herein below for an illustration purpose only; they are not limitative of the present invention:

FIG. 1 is a flow chart which schematically shows a known process for producing lactic acid;

FIG. 2 is a flow chart which schematically shows a process for producing D-type lactic acid according to an embodiment of the present invention; and,

FIG. 3 is a schematic diagram which shows a process for producing D-type lactic acid with the use of an active carbon stick according to an embodiment of the present invention.

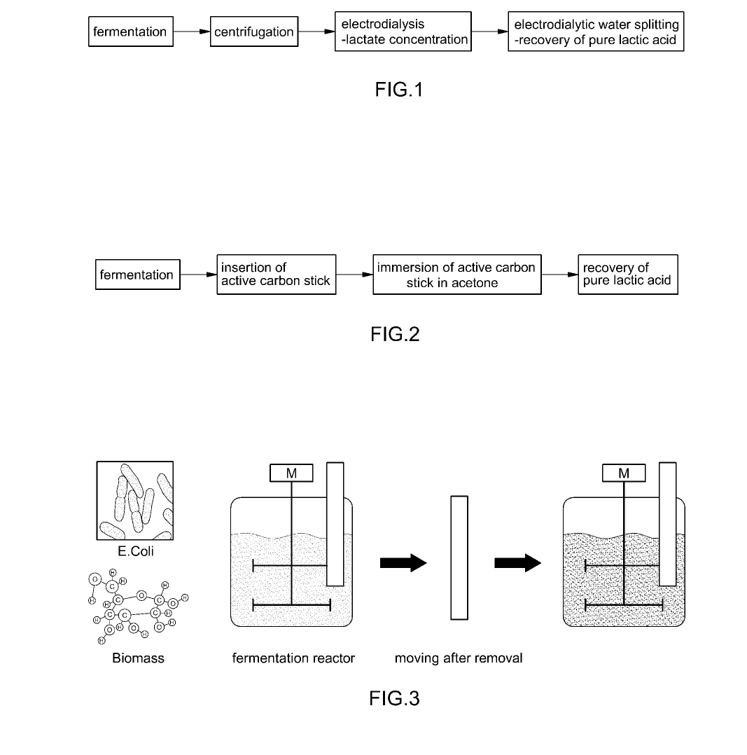

It should be understood that the drawings are not necessarily to scale, presenting a simplified representation of various features that are illustrative of the basic principles of the invention. The specific features of the present invention as disclosed herein, including, for example, specific dimensions, orientations, locations, and shapes can be determined in part by a particular intended application and a use environment.

In the figures, reference numbers refer to the same or equivalent parts of the present invention throughout the several figures of the drawing.

### DETAILED DESCRIPTION

Hereinafter reference is made in detail to various embodiments of the present invention, and examples of which are illustrated in the accompanying drawings and described below. While the invention is described in conjunction with the exemplary embodiments, it is understood that present description is not intended to limit the invention to those embodiments. On the contrary, the invention is intended to cover not only the exemplary embodiments, but also various alternatives, modifications, equivalents and other embodiments, which may be included within the spirit and scope of the invention and within the scope of the appended claims.

The present invention provides an efficient method for producing D-type lactic acid through a sugar fermentation process.

In the present invention, the sugar component is subject to microorganism fermentation followed by culturing in the fermentation reactor thereby to produce the fermentation liquor. The sugar component is selected from the group consisting of glucose, fructose, mannose, galactose and sucrose, or a mixture thereof. In a certain embodiment, the sugar component used herein is glucose.

In a particular embodiment, the microorganism is selected from E. coli and Lactobacillus and a mixture thereof. In one embodiment of the invention, the fermentation is performed by a batch process or a continuous process at a temperature of between 37° C. and 45° C. and at a pH of between 6.0 and 6.5.

If the pH in the fermentation reactor drops to between 1 and 5.5 due to the production of lactic acid therein, a sterilized active carbon stick is inserted into the fermentation reactor to absorb lactic acid produced. In certain embodiments, the active carbon stick is of a granule or a pellet type. The pore size of the active carbon stick is at a micrometer level, for example, within the range of between 0.1 μm and 0.5 μm. In one particular embodiment, the volume of the active carbon stick is 10˜20% of the volume of the fermentation liquor. In another embodiment, the active carbon stick is immersed in the fermentation reactor for a period time of between 30 minutes and 1 hour (that is, the dwell time).

Once the pH reaches a value of between 5.5 and 6.5 after keeping the active carbon stick in the fermentation reactor for a certain period of time to absorb lactic acid, the active carbon stick is removed from the fermentation reactor and then immersed into a recovery reactor.

The active carbon stick having lactic acid absorbed is subject to a detachment process during which lactic acid is dissolved into acetone in the recovery reactor. Once the pH value in the recovery reactor drops to between 1 and 5.5, the active carbon stick is removed from the recovery reactor. In one embodiment, the retention time for the active carbon stick in the recovery reactor is within the range of from 30 minutes to 1 hour. D-type lactic acid that is detached from the active carbon stick in the recovery reactor can be separated and recovered from acetone, which is a volatile organic solvent. In accordance with the invention, D-type lactic acid can be obtained in the high yield, for example, 98% or higher, and can be recovered in the form of a monomer.

Moreover, the active carbon stick, from which lactic acid is detached, can be sterilized and reinserted into the fermentation reactor to repeat all the steps above described.

According to the present invention, lactic acid produced in the fermentation reactor can be absorbed into the active carbon stick and then discharged as D-type lactic acid, that can be used as a starting material for producing poly-lactic acid through fermentation. The present invention thus offers advantages, such as, the acidity in the fermentation reactor is lowered, thereby to reduce adverse effects associated with growth and maintenance of the population of microorganism for the fermentation use, and further to produce D-type lactic acid with a high yield. Additionally, for the entire process of the poly-lactic acid production (the fermentation→the separation and purification→the lactide conversion→the resin polymerization), the manufacturing cost associated with the fermentation and the separation/purification steps can be significantly reduced.

The present invention is described herein below in detail with reference to examples. However, the present invention is not limited by the examples.

### EXAMPLES

The following examples illustrate the invention, but are not intended to limit the same.

The microorganism used herein was E. coli (KCTC 2223). The medium composition used for culturing microorganism seed contained 5.0 g of tryptone and 2.5 g of yeast extract in 1 L of an aqueous solution. After adding the bacterial strain to the medium with the above composition, the strain was subject to about 5 to 6 hours of the cultivation to produce the seed culturing-medium. The fermentation condition for producing D-type lactic acid contained 100 g/L of glucose, and all the media and containers used for the fermentation were sterilized at 120° C. for 20 minutes prior to the experiment.

The medium composition used in the main fermentation contained 50 g of glucose, 5 g of yeast extract, 0.5 g of sulfuric magnesium, 0.1 g of an anti-foam agent in 1 L of an aqueous solution. The bacterial strain was inoculated in the fermentation reactor to a concentration of 5% (v/v) of the seed culture-medium. The main fermentation reaction was performed at 37° C. The fermentation reaction was performed under an anaerobic condition and the pH was maintained at 6.0˜6.5.

A cylindrical active carbon stick, having a pore size of about 0.2 micrometer, was first sterilized by wrapping it in a fiber type packing material and dipping the wrapped material in a sterilizer at 120° C. for about 10 minutes. When the acidity in the fermentation reactor reached a pH value between 1 and 5.5 due to the production of lactic acid, the active carbon stick was dipped into the fermentation reactor and dwelled there for a period between about 30 minutes and 1 hour. When the pH value in the fermentation reactor reached between 5.5 and 6.5, the active carbon stick with lactic acid absorbed therein was pulled out of the fermentation reactor. The active carbon stick was then dipped into the recovery reactor containing 70 wt % of acetone and dwelled there for a period between 30 minutes and 1 hour. The mixture solvent was stirred by a stirrer in the reactor. Lactic acid absorbed in the active carbon stick was then detached to get into the mixture solvent to recover D-type lactic acid with an acidic pH of between 1 and 5.5. The active carbon stick was recycled after the sterilization process. The concentration of D-type lactic acid and the glucose component in the fermentation liquor were analyzed by High Performance Liquid Chromatography (HPLC).

The method for producing D-type lactic acid according to the present invention has achieved a production of about 60 g of D-type lactic acid per 100 g of glucose, with the produced D-type lactic acid at the purity of 98.1 wt %.

The invention has been described in detail with reference to embodiments thereof. However, it will be appreciated by those skilled in the art that changes may be made in these embodiments without departing from the principles and spirit of the invention, and the scope of the appended claims and their equivalents.

What is claimed is:

1. Method for producing D-type lactic acid through a sugar fermentation, comprising steps of:
(a) adding a microorganism to a sugar component in a fermentation reactor to produce a fermentation liquor through fermentation;
(b) inserting a sterilized active carbon stick in said fermentation reactor to absorb lactic acid produced through the fermentation;
(c) removing from the fermentation reactor said active carbon stick with lactic acid absorbed therein and immersing said active carbon stick in a recovery reactor; and,
(d) detaching lactic acid from the active carbon stick using acetone in the recovery reactor, thereby to produce D-type lactic acid.

2. A method for producing D-type lactic acid as claimed in claim 1, wherein said sugar component is selected from glucose, fructose, mannose, galactose and sucrose or a mixture thereof.

3. A method for producing D-type lactic acid as claimed in claim 1, wherein said microorganism is selected from E. coli and Lactobacillus species or a mixture thereof.

4. A method for producing D-type lactic acid as claimed in claim 1, wherein the fermentation in said step (a) is performed at a temperature ranging between 37° C. and 45° C. and a pH ranging between 6.0 and 6.5.

5. A method for producing D-type lactic acid as claimed in claim 1, wherein the fermentation in said step (a) is performed by a batch process or a continuous process.

6. A method for producing D-type lactic acid as claimed in claim 1, wherein said step (b) is performed at pH 1˜5.5.

7. A method for producing D-type lactic acid as claimed in claim 1, wherein said active carbon stick is of a granule type or a pellet type.

8. A method for producing D-type lactic acid as claimed in claim 1, wherein said active carbon stick has a pore size ranging between 0.1 μm and 0.5 μm.

9. A method for producing D-type lactic acid as claimed in claim 1, wherein the volume of said active carbon stick in step (b) ranges from 10% to 20% of the volume of the fermentation liquor.

10. A method for producing D-type lactic acid as claimed in claim 1, wherein step (c) is performed at pH 5.5˜6.5.

11. A method for producing D-type lactic acid as claimed in claim 1, wherein step (d) is performed at pH 1˜5.5.

12. A method for producing D-type lactic acid as claimed in claim 1, wherein D-type lactic acid is produced in a monomer state in said step (d).

13. A method for producing D-type lactic acid as claimed in claim 1, wherein the dwell time of the active carbon stick in steps (b) and (d) ranges between 30 minutes and 1 hour.

14. A method for producing D-type lactic acid as claimed in claim 1, wherein it further comprises steps of sterilizing the active carbon stick with lactic acid detached in step (d) and then adding the sterilized active carbon stick into the fermentation reactor.
